(19) United States
(12) Patent Application Publication
(10) Pub. No.: US 2020/0376147 A1
(43) Pub. Date: Dec. 3, 2020

(54) SODIUM SACCHARIN HAVING CONJUGATED LIGAND, DERIVATIVES THEREOF AND PROCESS FOR THE PREPARATION THEREOF

(71) Applicant: KYUNG-IN SYNTHETIC CORPORATION, Incheon (KR)

(72) Inventors: Donggil Kim, Jeju-do (KR); Eui Jae Lee, Gyeonggi-do (KR)

(21) Appl. No.: 16/423,448

(22) Filed: May 28, 2019

(51) Int. Cl. A61K 51/04; A61K 51/12; G01N 33/574

(52) U.S. Cl. CPC A61K 51/0482; G01N 33/574; A61K 51/121; A61K 51/0497

(57) ABSTRACT

The present invention provides sodium saccharin bound with a ligand, derivatives thereof, and a process for preparation thereof. According to the present invention, there is provided a compound which is specifically bonded to hypoxic tumors. Specifically, the compound having sodium saccharin (SAC) bound with a ligand compound, or derivatives thereof can be applied to a contrast agent for cancer diagnosis.

### BACKGROUND OF THE INVENTION

### 1. Field of the Invention

The present invention relates to sodium saccharin bound with a ligand, which is a substance having a selective targeting action on hypoxic tumors, derivatives thereof, and a process for preparation thereof.

### 2. Description of the Related Art

Among radio isotopes, a radio isotope [⁶⁸Ga] gallium emitting positron, principally can be obtained from extraction by the collapse according to physical half-life (270.95 days) of [⁶⁸Ge] germanium which is a parent nuclide, by a generator and it can be easily coordinated to a ligand having negative charge (Non-Patent Document 1). The radioisotope [⁶⁸Ga] gallium has long been used for diagnosis and treatment of tumors by marking biologically active molecules used for cancer cell targets.

About 90% of all currently known cancers are solid tumors. Concentrations of oxygen in solid tumors are very variable. Continued oxygen deficiency occurs necrosis, but viable tumor cells surround necrotic tissues and become hypoxic. Hypoxic tumor cells are characterized by their ability to resist glycolysis, angiogenesis, proliferation, metastasis, invasion and radiation therapy by altering their gene transcription patterns to overcome stress caused by oxygen deficiency.

Carbonic Anhydrase IX (CA IX) is present on the surface of hypoxic tumor cells. When cancer cells use glucose as an energy source in a state of oxygen deficiency, pyruvic acid is produced as a byproduct, which promotes conversion of aerobic respiration to anaerobic respiration. In this case, lactic acid accumulates, and therefore pH in the tumor rapidly decreases. Carbonic Anhydrase IX (CA IX) plays a large role in neutralizing carbon dioxide (CO₂) by using bicarbonate (HCO₃⁻) and maintaining the survival of cancer cells by regulating pH in the tumor cells (Non-Patent Document 2).

In addition, Carbonic Anhydrase IX (CA IX) is expressed only in hypoxic tumor cells, not normal cells, and saccharin (SAC) selectively binds to Carbonic Anhydrase IX without affecting surrounding normal cells, thereby inhibiting activity. Thus, studies on inhibition of proliferation of tumor cells due to such binding characteristics have been reported. A representative result of the study is an effective inhibition of cancer cell proliferation in the evaluation of anti-proliferative effect of saccharin on various cancer cell lines and MSCs by SW Kim Group of Korea University (Non-Patent Document 3).

The present invention is to develop a molecular imaging agent for tumor diagnosis using biological characteristics of tumor as described above.

### PRIOR ART DOCUMENT

1. Velikyan I. ⁶⁸Ga-Based Radiopharmaceuticals: Production and Application Relationship. Molecules Review. 2015. 12913-12943.

2. Mahon B P, Hendon A M, Driscoll J M, Rankin G M, Poulsen S A, Supuran C T, McKenna R. Saccharin: a lead compound for structure-based drug design of carbonic anhydrase IX inhibitors. 2015. 849-854.

3. JS Choi, Sang Yong Park, Man Gil Dong Beom Lee, Tae Bok Lee, Ji Hye Heo, Min Woo Lee, and Suhng Wook Kim Estimation of Anti-proliferative Activity of Saccharin against Various Cancer Cell Lines and MSCs. 2016. 169-175.

### SUMMARY OF THE INVENTION

It is an object of the present invention to provide a compound having sodium saccharin (SAC) bound with a ligand which has high binding affinity for Carbonic Anhydrase IX (CA IX) in hypoxic tumors, and derivatives thereof.

It is also an object of the present invention to provide a method for producing the above compound and derivatives thereof.

It is also an object of the present invention to provide a contrast agent comprising the above compound and derivatives thereof.

In order to solve the above problems, the present invention provides a compound having sodium saccharin (SAC) bound with a ligand compound, and derivatives thereof.

According to one embodiment, the ligand compound may comprise 1,4,7-triazacyclononane-1,4,7-triacetic acid (NOTA).

According to one embodiment, the compound may comprise the structure as shown in formula 1.

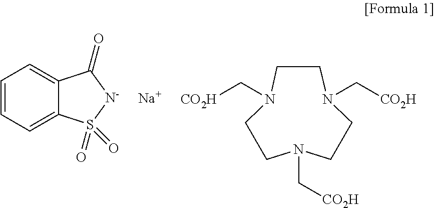 [SMILES: O=C1[N-]S(=O)(=O)c2ccccc21 ; [Na+] ; O=C(O)CN1CCN(CC(=O)O)CCN(CC(=O)O)CC1]

According to one embodiment, the compound may comprise the structure as shown in formula 2.

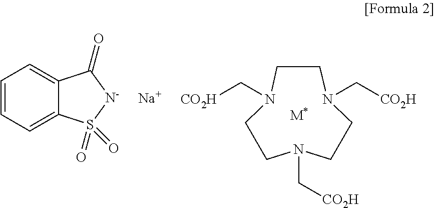 [SMILES: O=C1[N-]S(=O)(=O)c2ccccc21 ; [Na+] ; O=C(O)CN1CCN(CC(=O)O)CCN(CC(=O)O)CC1]

In the formula 2,

Sodium saccharin (SAC) is ionically bonded to 1,4,7-triazacyclononane-1,4,7-triacetic acid (NOTA) and M* is a radio isotope which is coordinated to the NOTA.

According to one embodiment, the radio isotope may be ⁶⁸Ga.

According to another embodiment of the present invention, there is provided a method for producing the above compound and derivatives thereof, the method comprising the steps of:

ionically binding sodium saccharin (SAC) with a ligand compound to prepare a sodium saccharin-ligand compound, and

reacting the sodium saccharin-ligand compound with a radio isotope.

According to one embodiment, the ligand compound may comprise 1,4,7-triazacyclononane-1,4,7-triacetic acid (NOTA).

According to one embodiment, the radio isotope may be ⁶⁸Ga.

According to another embodiment of the present invention, there is provided a contrast agent comprising the above compound and derivatives thereof.

In addition, the contrast agent can be used in Positron Emission Tomography (PET) for cancer diagnosis.

Other specific embodiments of the present invention are included in the following detailed description.

### EFFECT OF THE INVENTION

The present invention relates to sodium saccharin having conjugated ligand, derivatives thereof, and a process for preparation thereof. According to the present invention, there is provided a compound which is specifically bonded to hypoxic tumors, or derivatives thereof. Further, by applying the above compound or derivatives thereof to a contrast agent for cancer diagnosis, it is possible to effectively diagnose the tumor by improving the discrimination.

### BRIEF DESCRIPTION OF THE DRAWINGS

FIG. 1 is a graph showing the result of thin-layer chromatography (TLC) on a compound according to the present invention.

FIG. 2 is an image of positron emission computed tomography (PET/CT) using a compound according to the present invention.

FIG. 3 is a PET/CT image for identification of tumor selectivity.

FIG. 4 is a graph showing the distribution of a compound according to the present invention in tissues.

### DETAILED DESCRIPTION OF THE INVENTION

Since various modifications and variations can be made in the present invention, particular embodiments are illustrated in the drawings and will be described in detail in the detailed description. It should be understood, however, that the invention is not intended to be limited to the particular embodiments, but includes all modifications, equivalents, and alternatives falling within the spirit and scope of the invention. In the following description of the present invention, detailed description of known functions will be omitted if it is determined that it may obscure the gist of the present invention.

Hereinafter, a compound or derivatives thereof according to an embodiment of the present invention will be described in more detail.

The present invention provides a compound having sodium saccharin (SAC) bound with a ligand compound, or derivatives thereof.

According to one embodiment, the ligand compound may comprise 1,4,7-triazacyclononane-1,4,7-triacetic acid (NOTA). Such a metal affinity ligand compound can prevent radio isotopes from liberating in the body, thereby releasing the radioactive substance out of the body. From this, it can serve as a protecting agent by reducing cytotoxicity (Marouan Rami et al., Carbonic anhydrase inhibitors: Gd(III) complexes of DOTA- and TETA-sulfonamide conjugates targeting the tumor associated carbonic anhydrase isozymes IX and XII , New J. Chem., 2010, 34, 2139-2144; Silvio Aime et al., NMR relaxometric studies of Gd(III) complexes with heptadentate macrocyclic ligands, Magnetic Resonance in Chemistry (1998) Volume: 36, Issue: 51, Pages: S200-S208).

Specifically, the compound according to the present invention may comprise the compound of the following formula 1.

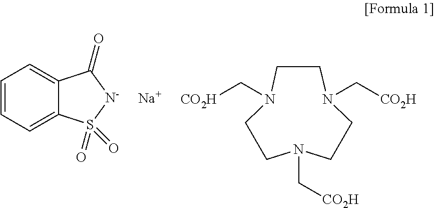 [SMILES: O=C1[N-]S(=O)(=O)c2ccccc21 ; [Na+] ; O=C(O)CN1CCN(CC(=O)O)CCN(CC(=O)O)CC1]

In the above formula 1, sodium of saccharin and a carboxyl group of 1,4,7-triazacyclononane-1,4,7-triacetic acid (NOTA) are bonded to each other.

According to one embodiment, the compound according to the present invention may comprise the structure of the following formula 2.

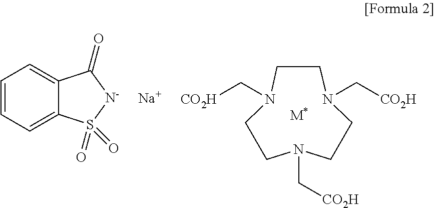 [SMILES: O=C1[N-]S(=O)(=O)c2ccccc21 ; [Na+] ; O=C(O)CN1CCN(CC(=O)O)CCN(CC(=O)O)CC1]

In the formula 2,

Sodium saccharin (SAC) is ionically bonded to 1,4,7-triazacyclononane-1,4,7-triacetic acid (NOTA), and

M* is a radio isotope which is coordinated to the NOTA.

According to one embodiment, the radio isotope may be ⁶⁸Ga.

The NOTA can coordinate with the radio isotope to easily form a complex.

That is, as described above, the present invention can provide a compound having conjugated ligand in order to bind a radio isotope to sodium saccharin (SAC) stably.

According to other embodiment of the present invention, there is provided a method for producing a compound and derivatives thereof, the method comprising the steps of:

ionically binding sodium saccharin (SAC) with a ligand compound to prepare a sodium saccharin-ligand compound, and

reacting the sodium saccharin-ligand compound with a radio isotope.

According to one embodiment, the method of the present invention may comprise a step of reacting a solution of ⁶⁸Ga with a buffer solution of pH 5 to 6 and a compound of formula 1, for example, at 50 to 100° C., for example, at 80° C. to form a compound of formula 2. The buffer solution may include, but is not limited to, for example, sodium acetate.

According to one embodiment, the ligand compound may comprise 1,4,7-triazacyclononane-1,4,7-triacetic acid (NOTA).

Specifically, the production process according to one embodiment of the present invention is shown in Scheme 1.

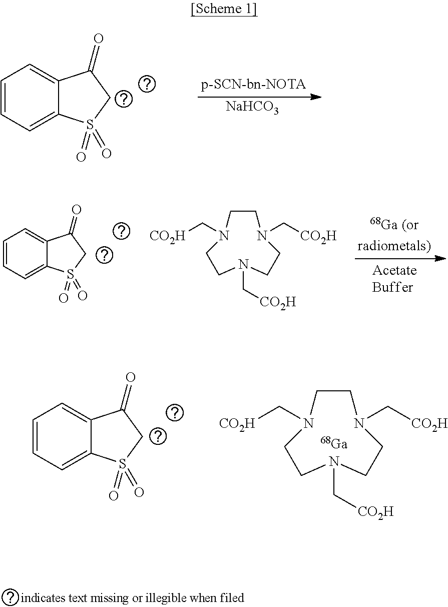

As shown in Scheme 1, in the present invention, a ligand was used to coordinate sodium saccharin with a metallic radio isotope. The ligand compound includes a compound in which sodium of saccharin are bonded to a carboxyl group of 1,4,7-triazacyclononane-1,4,7-triacetic acid (NOTA).

Sodium saccharin (SAC) has a property of specifically binding to carbonic anhydrase IX (CA IX), which is not expressed in normal cells but expressed only in hypoxic tumors. Sodium saccharin (SAC) is labeled with a radio isotope through the ligand compound. Due to such targeting to hypoxic tumors, the substance according to the present invention can be applied to a contrast agent.

Specifically, a contrast agent comprising a compound according to the present invention or derivatives thereof can be used as a contrast agent of Positron Emission Tomography (PET) for hypoxic tumors.

According to one embodiment, the PET contrast agent according to the present invention can be formulated to an injection or the like.

According to one embodiment, when the compound according to the present invention is used as a contrast agent, a non-toxic solution, which is an isotonic component with blood, may be contained as a diluent. The diluent may include, for example, sodium chloride solution, potassium chloride solution, sodium bicarbonate solution, Hartmann's solution, glucose solution, glucose physiological saline solution and the like. Specific examples of the diluent include phosphate buffer solution of pH 7.4 or the like.

Hereinafter, embodiments of the present invention will be described in detail so that those skilled in the art can easily carry out the present invention. The present invention may, however, be embodied in many different forms and should not be construed as limited to the embodiments set forth herein.

### EXAMPLE 1

### Preparation of Compound of Formula 1 (SAC-NOTA)

To prepare a compound of formula 1 (SAC-NOTA), a K₂CO₃ solution and 1 mg of a ligand compound, 1,4,7-triazacyclononane-1,4,7-triacetic acid (NOTA) were added to a reaction vessel and dissolved therein. In another vessel, 1.83 mg (5 eq.) of sodium saccharin (SAC) was dissolved in distilled water (D.W) and then added to the reaction vessel. After stirring at room temperature for 24 hours, separation and purification were carried out using a column (Pharmacia Fine Chemicals, Sephadex G-25).

### EXAMPLE 2

### Preparation of Compound of Formula 2 (SAC-NOTA-⁶⁸Ga)

To prepare a compound of formula 2 (SAC-NOTA-⁶⁸Ga), gallium was used as a radio isotope. ⁶⁸Ga which was extracted from a ⁶⁸Ga generator was obtained as a colorless transparent solution dissolved in a dilute hydrochloric acid solution. After taking it properly according to the amount of radioactivity required for the experiment (above 0.1 mCi), the ⁶⁸Ga solution was placed in a suitable glass vessel and dried at 100° C. while blowing nitrogen therein. The dried ⁶⁸Ga is applied to the glass vessel in the form of a transparent film. 0.5 mL of the compound of formula 1 (SAC-NOTA) which was isolated and extracted in Example 1 was added to the reaction vessel coated with ⁶⁸Ga, and then the labeling reaction was allowed to proceed for 10 minutes at pH 5 to 6 at 80° C. Reaction according to Example 2 is shown in Scheme 2.

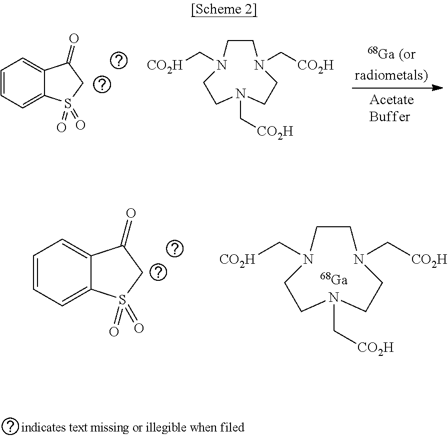

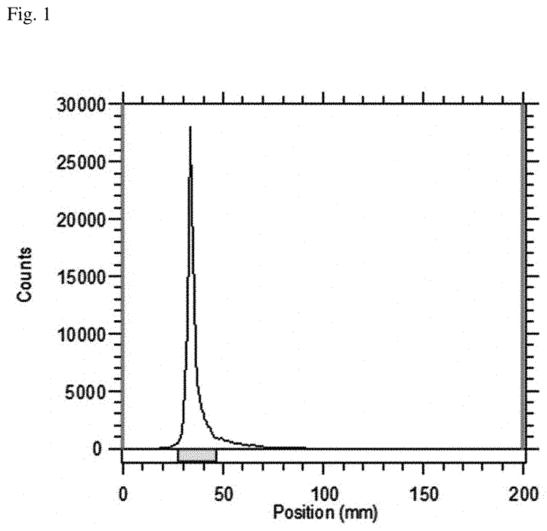

The final compound, the compound of formula 2 (SAC-NOTA-⁶⁸Ga), was adsorbed on a thin layer chromatography (TLC) plate and sufficiently developed with 0.1 M citrate buffer solution. From this, radiochemical yield and purity was identified. The results are shown in FIG. 1. FIG. 1 is a graph showing Radio-TLC (AR-2000, Eckert & Ziegler) results of the compound of formula 2 (SAC-NOTA-⁶⁸Ga).

As shown in FIG. 1, the radiochemical yield and purity from the TLC image are confirmed to be 98% or more on average.

### EXPERIMENTAL EXAMPLE 1

### Evaluation of Stability

In order to evaluate stability for the compound of formula 2 (SAC-NOTA-⁶⁸Ga) prepared in Example 2, stability in human serum and mouse serum was measured. 100 μL of 0.4 mCi of the compound of formula 2 (SAC-NOTA-⁶⁸Ga) was mixed with 0.5 mL of each of human serum and mouse serum, and then cultured at 37° C. Thin layer chromatography (TLC) was performed using radio-thin film chromatography (AR-2000, Eckert & Ziegler) equipment at the time to be measured (1 hour, 3 hours, 6 hours). The measurement results of stability of the compound of formula 2 (SAC-NOTA-⁶⁸Ga) in the serum over time are shown in Table 1 below.

TABLE 1

| | | | |
|---|---|---|---|
| Item | 1 h | 3 h | 6 h |

| | | | | |
|---|---|---|---|---|
| Compound of | Human serum | 100% | 100% | 98% |
| formula 2 | Mouse serum | 100% | 98.26% | 98.50% |

As shown in Table 1, since the form of the compound of formula 2 as a target compound is maintained, that is, the purity is maintained, it can be confirmed that the compound of formula 2 is stable for 6 hours or longer in human serum and mouse serum.

### EXPERIMENTAL EXAMPLE 2

### PET Image Acquisition of SAC-NOTA-⁶⁸Ga

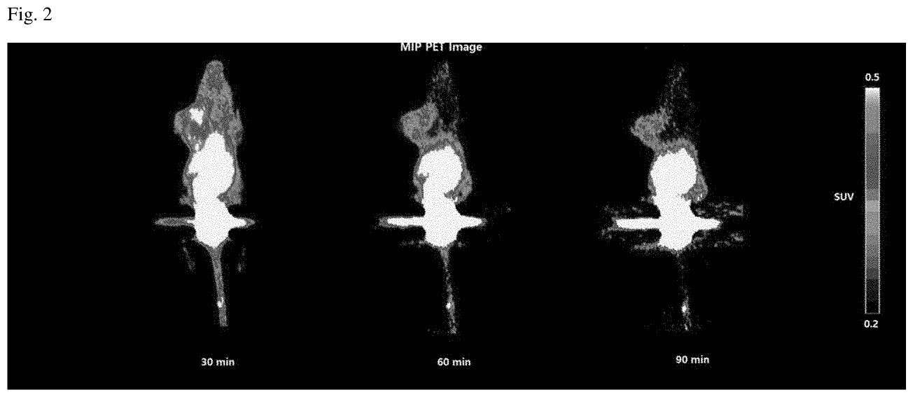

Positron emission tomography (PET) images were taken with PET/CT equipment for small animals (INVEON, Simens Medical Solutions). U87MG cells, a malignant glioma cell line, were implanted subcutaneously in the left shoulder of a nude mouse to form a tumor model. PET/CT images were obtained at 30 minutes, 60 minutes, and 90 minutes by injecting 0.23 mCi of the compound of formula 2 (SAC-NOTA-⁶⁸Ga) prepared in Example 2 into the tail vein of the tumor-transplanted mouse (nu/nu nude, 20 g). The results were shown in FIG. 2. As shown in FIG. 2, it can be observed that the signal increases at the tumor site after administration of the compound of formula 2 (SAC-NOTA-⁶⁸Ga).

### EXPERIMENTAL EXAMPLE 3

### Identification of Tumor Selectivity of SAC-NOTA-⁶⁸Ga

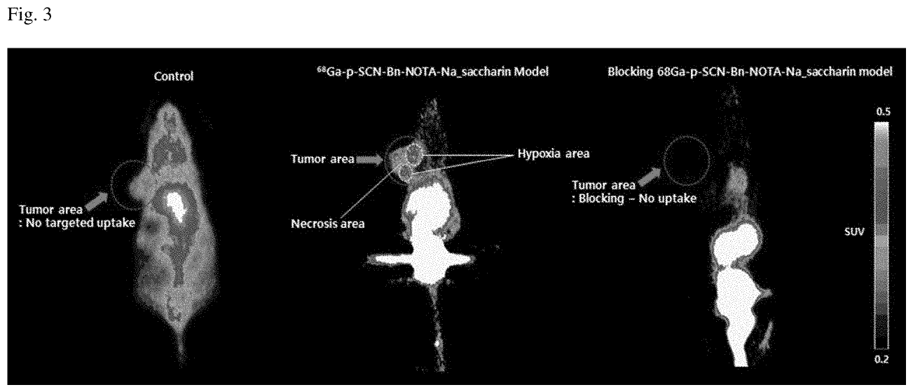

Experiments were carried out to identify selective specificity for receptors expressed in tumor cells of SAC-NOTA-⁶⁸Ga prepared in Example 2. U87MG cells, a malignant glioma cell line, were injected subcutaneously into the shoulder of a nude mouse to form a tumor model. First, sodium saccharin (10 mg/kg) was injected through the tail vein of the anesthetized mouse to block the receptor. After 30 minutes, 0.2 mCi of the SAC-NOTA-⁶⁸Ga was injected to acquire PET/CT images in the same manner as in Experimental Example 2. The results were shown in FIG. 3.

In FIG. 3, a PET/CT image (center) is taken by injecting SAC-NOTA-⁶⁸Ga without blocking the receptor present in the tumor and a PET/CT image (right) is taken by injecting SAC-NOTA-⁶⁸Ga after blocking the receptor present in the tumor by injecting sodium saccharin.

According to FIG. 3, it can be seen that the signal of the tumor site where the receptor is blocked is lower than the signal of the tumor site where the receptor is not blocked. From these results, it can be seen that SAC-NOTA-⁶⁸Ga has selective specificity for receptors expressed in the tumor cell.

### EXPERIMENTAL EXAMPLE 4

### Identification of Distribution of SAC-NOTA-⁶⁸Ga in Tissues

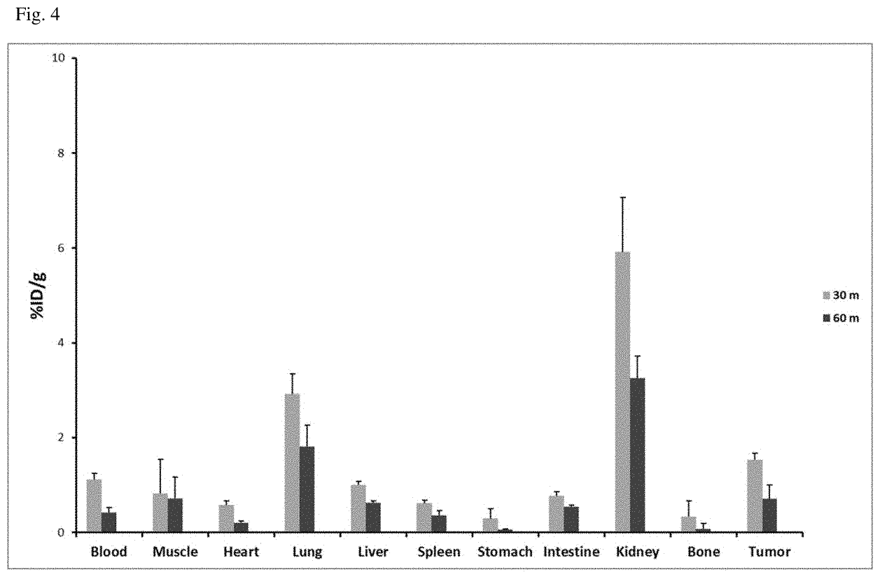

The distribution in mouse tissues of SAC-NOTA-⁶⁸Ga prepared in Example 2 was confirmed. U87MG cells, a malignant glioma cell line, were injected subcutaneously into the shoulder of a nude mouse (body weight 20 g) to form a tumor model. 0.01 mCi of SAC-NOTA-⁶⁸Ga was injected through the tail vein of the tumor-bearing mouse (n=4). After 30 minutes and 60 minutes of injection, the organs (blood, muscle, heart, lung, liver, spleen, stomach, intestine, kidney, bone, tumor) were extracted, respectively. The radioactivity of the tissue was measured with a gamma counter. The results were shown in FIG. 4.

FIG. 4 is a graph showing a ratio of the tissue radioactivity to the amount of injection (% ID/g) measured in various organs after injection of SAC-NOTA-⁶⁸Ga over time.

From FIG. 4, it can be seen that the radioactivity in the tumor is maintained at 1.52% after 30 minutes of injection and at 0.69% after 60 minutes of injection. In addition, it can be seen that the radioactivity value of the liver tissue was kept low at 0.99% after 30 minutes and at 0.63% after 60 minutes of injection.

The above description is only illustrative of the technical idea of the present invention, and various changes and modifications may be made by those skilled in the art without departing from the essential characteristics of the present invention. It should be noted that the embodiments disclosed in the present invention are not intended to illustrate rather than limit the scope of the present invention. The scope of the present invention is not limited by these embodiments. The scope of protection of the present invention should be construed according to the following claims, and all technical ideas within the scope of equivalents should be construed as falling within the scope of the present invention.

What is claimed is:

1. A compound having sodium saccharin (SAC) bound with a ligand compound, or derivatives thereof.

2. The compound or derivatives thereof according to claim 1, wherein the ligand comprises 1,4,7-triazacyclononane-1,4,7-triacetic acid (NOTA).

3. The compound or derivatives thereof according to claim 1, wherein the compound comprises the structure of the following formula 1:

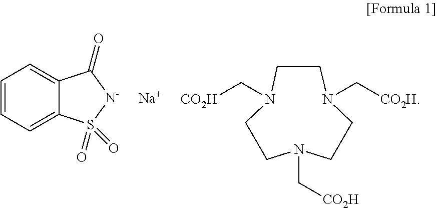 [SMILES: O=C1[N-]S(=O)(=O)c2ccccc21 ; [Na+] ; O=C(O)CN1CCN(CC(=O)O)CCN(CC(=O)O)CC1]

4. The compound or derivatives thereof according to claim 3, wherein the compound comprises the structure of the following formula 2: 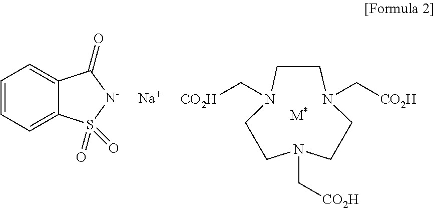 [SMILES: O=C1[N-]S(=O)(=O)c2ccccc21 ; [Na+] ; O=C(O)CN1CCN(CC(=O)O)CCN(CC(=O)O)CC1]
in the formula 2,
Sodium saccharin (SAC) is ionically bonded to 1,4,7-triazacyclononane-1,4,7-triacetic acid (NOTA) and
M* is a radio isotope which is coordinated to the NOTA.

5. The compound or derivatives thereof according to claim 4, wherein the radio isotope is ⁶⁸Ga.

6. A method for producing a compound or derivatives thereof, the method comprising the steps of:
ionically binding sodium saccharin (SAC) with a ligand compound to prepare a sodium saccharin-ligand compound, and
reacting the sodium saccharin-ligand compound with a radio isotope.

7. The method for producing a compound or derivatives thereof according to claim 6, wherein the ligand compound comprises 1,4,7-triazacyclononane-1,4,7-triacetic acid (NOTA).

8. The method for producing a compound or derivatives thereof according to claim 6, wherein the radio isotope is ⁶⁸Ga.

9. A contrast agent comprising the compound according to claim 1 or derivatives thereof.

10. The contrast agent according to claim 9, wherein the contrast agent is used in Positron Emission Tomography (PET) for cancer diagnosis.
